(19) United States
(12) Patent Application Publication
(10) Pub. No.: US 2019/0161431 A1
(43) Pub. Date: May 30, 2019

(54) APPARATUS AND METHOD FOR CONTINUOUS PRODUCTION OF POLYETHYLENE GLYCOL DINITRATE

(71) Applicant: Avocet IP Limited, Berwick upon Tweed (GB)

(72) Inventors: James Robert JENNINGS, Yarm (GB); Glyn David SHORT, Hockessin, DE (US)

(21) Appl. No.: 16/265,333

(22) Filed: Feb. 1, 2019

Related U.S. Application Data
(63) Continuation-in-part of application No. 15/551,108, filed on Aug. 15, 2017, now Pat. No. 10,232,341.

(30) Foreign Application Priority Data
Feb. 15, 2015 (GB) 1502523.2

(51) Int. Cl. C07C 205/02; B01D 11/04; B01D 1/06

(52) U.S. Cl. CPC C07C 205/02; B01D 11/0492; B01D 2257/404; B01D 1/065; B01D 2202/00; B01D 11/0488

(57) ABSTRACT

A reaction apparatus for producing polyethylene glycol dinitrate (PEGDN) in a continuous manner includes a series of reaction cells spatially disposed in one or more planar structures and a separation arrangement for separating PEGDN and Ammonium Nitrate, in a continuous manner. The separation arrangement is a thin film evaporator and/or falling film evaporator. The plurality of reaction cells includes a feed preparation section having feedstreams for continuously providing an acid composition and a glycol composition to reaction cells. The plurality of reaction cells further includes a nitration section, where the acid composition and the glycol composition react to generate a reaction composition, and a quench and neutralization section, having feed for cooling arrangement and a plurality of feeds for providing an alkaline composition to at least partially neutralize reaction composition. The acid composition includes a mixture of dilute nitric acid and concentrated sulphuric acid.

### TECHNICAL FIELD

The present disclosure generally relates to fuel additives, for example polyethylene glycol (PEG) or polyethylene glycol dinitrate (PEGDN), to be used in conjunction with combustible hydrocarbon fuels. Specifically, the present disclosure relates to an apparatus and a method for producing aforementioned fuel additives using a continuous process.

### BACKGROUND

It is contemporary practice to combust fuels together with fuel additives in cylinders of internal combustion engines, wherein the fuel additives assist to protect the engines from oxidative corrosion, as well as providing a degree of lubrication and cetane control. In certain engines, a given fuel includes an additive, such that the given fuel and the additive are injected through a same nozzle of a given cylinder; conversely, in other engines, an additive is injected separately to a hydrocarbon fuel into cylinders, by using multiple nozzles per cylinder.

Polyethylene glycol dinitrate (PEGDN) is a known additive for use with hydrocarbon fuels. Moreover, it is known practice to manufacture PEG nitrate in a two-step chemical process as provided in Table 1.

TABLE 1
Known PEGDN manufacturing process

| Step | Reaction |
|---|---|
| 1 | nC₂H₄O + H₂O → HO—(CH₂CH₂—O)ₙH |
| 2 | ROH + HNO₃ → R − O − N_{=O}^{=O} |

In Table 1, “R” represents a molecular grouping including ethylene glycol.

In a known publication “Organic Chemistry of Explosives (2007)” by P. Agrawal and R D. Hodgson, there is described a mixed acid generated from sulphuric and nitric acids, which still remains a most important reagent for the industrial production of nitrate esters:

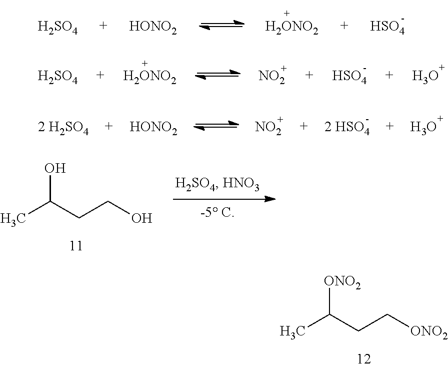

Generally, nitrations with mixed acid and nitric acid are exothermic. Therefore, on a large scale, there is always a potential problem of thermal runaway and an associated risk of explosion. Consequently, on an industrial scale, the mixed acid nitration of polyols requires strict control, including:

(i) remote handling;
(ii) elaborate reactors; and
(iii) blast-proof buildings.

Furthermore, conventional nitration usually follows a batch or a semi-batch approach, where reactants are mixed and the reaction itself are carried out very slowly. A continuous process has also been claimed by Corning Incorporated (USA), using their Advanced Flow Reactor. However, specifically for the production of PEGDN, some of the most important concerns, which do not allow for an easy scale up include: (i) an inadequate heat transfer area, (ii) an inhomogeneous system, mainly due to immiscible substrates and inefficient mixing, leading to mass transfer limitations, (iii) batch-to-batch variation in the degree of conversion, yield and selectivity, (iv) prolonged reaction times, (v) reactions at very low temperatures to reduce the rate of heat generation, (vi) the use of excess nitrating agent, mainly the spent acid, which occupies significant volume, has to be neutralized thereby needing large quantity of water, and generates inorganic salts.

Moreover, product separation may be a frequent problem associated with the mixed acid nitration of polyols. There arises a mixed acid residue from the method, and associated aqueous washings often contain considerable amounts of dissolved nitrate ester, presenting both a safety and a waste problem; ethylene glycol dinitrate is soluble in water to the extent of 0.5 g per 100 ml.

Therefore, there is a need for improved apparatus and methods of producing fuel additives, for example based on ethylene glycol nitrates, for example PEG, which address aforementioned problems more effectively.

### SUMMARY

The present disclosure seeks to provide an improved apparatus for producing nitrate esters, for example polyethylene glycol dinitrate.

According to an aspect, there is provided a reaction apparatus for producing polyethylene glycol dinitrate (PEGDN) in a continuous manner, wherein the reaction apparatus comprising:

a series of reaction cells spatially disposed in one or more planar structures, wherein the plurality of reaction cells include
a feed preparation section having feedstreams for continuously providing an acid composition and a glycol composition to reaction cells thereof, wherein the acid composition includes a mixture of dilute nitric acid and concentrated sulphuric acid,
a nitration section in which the acid composition and the glycol composition react in reaction cells in a continuous manner to generate a reaction composition, and
a quench and neutralization section having a feed for a cooling arrangement for cooling reaction cells to avoid spatial reaction hotspots and thereby preventing thermal runaway occurring within the reaction apparatus, and a plurality of feeds for providing an alkaline composition to at least partially neutralize the reaction composition to cause at least a portion of the polyethylene glycol dinitrate to deposit from a solution of the reaction composition; and
   a separation arrangement for separating polyethylene glycol dinitrate (PEGDN) and Ammonium Nitrate, in a continuous manner, wherein the separation arrangement is a thin film evaporator and/or falling film evaporator.

In one embodiment, the acid composition includes the dilute nitric acid in a concentration range of 50 to 70 weight %.

Optionally, the concentration of dilute nitric acid is 60 weight %.

Optionally, the acid composition includes the concentrated sulphuric acid in a concentration range of 96 to 98 weight %.

In one embodiment, the glycol composition includes PEG with a molecular weight in a range of 150 to 800.

Optionally, the reaction composition has a pH in a range of 4 to 12.

In one embodiment, the separation arrangement separates the PEGDN and Ammonium Nitrate using a hydrophobic solvent.

Optionally, the hydrophobic solvent is one of methylene chloride, a hexane, a pentane or a silicone.

In one embodiment, the feed for the cooling arrangement uses a coolant applied to a region which is spatially adjacent to the series of reaction cells.

Optionally, the series of reaction cells are cooled in operation using a coolant at a temperature in a range of 0° C. to 15° C.

Optionally, the acid composition includes the dilute nitric acid and the concentrated sulphuric acid in equal volumes.

The apparatus of the present disclosure is of advantage in having a smaller inventory giving better temperature control, good heat transfer enabling quicker mixing of reactants, potential to operate at higher temperature to further increase rate, on-line neutralisation, short residence time improving selectivity and yield, lower capital and ease of automation.

It will be appreciated that features of the disclosure are susceptible to being combined in various combinations without departing from the scope of the disclosure as defined by the appended claims.

### DESCRIPTION OF THE DIAGRAMS

Embodiments of the present disclosure will now be described, by way of example only, with reference to the following diagrams wherein:

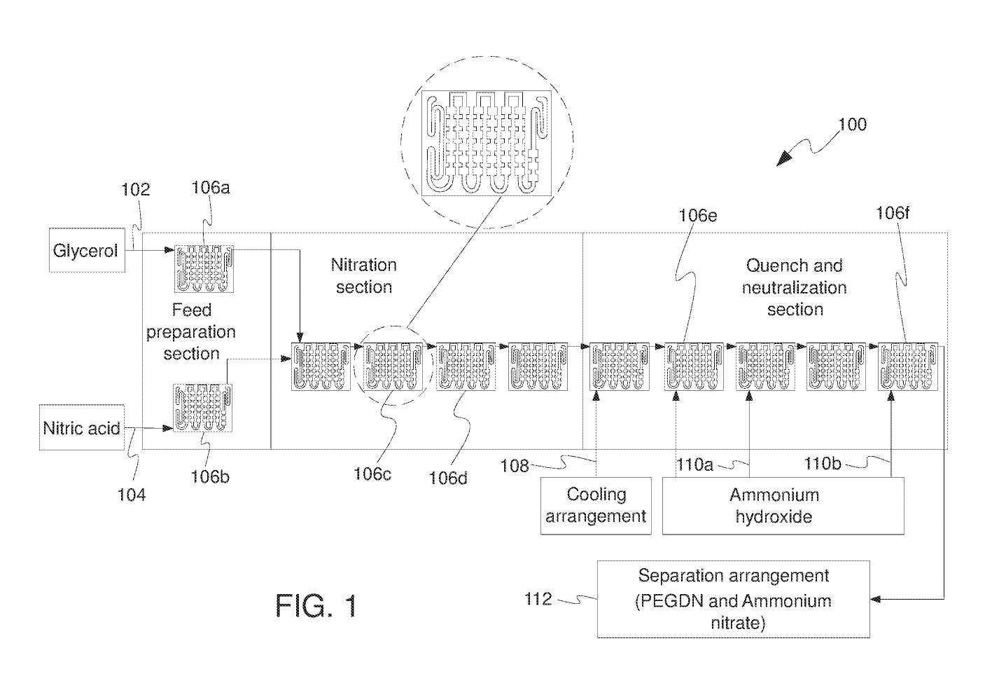

FIG. 1 is an illustration of an apparatus for producing PEGDN and ammonium nitrate, according to an embodiment of the present disclosure;

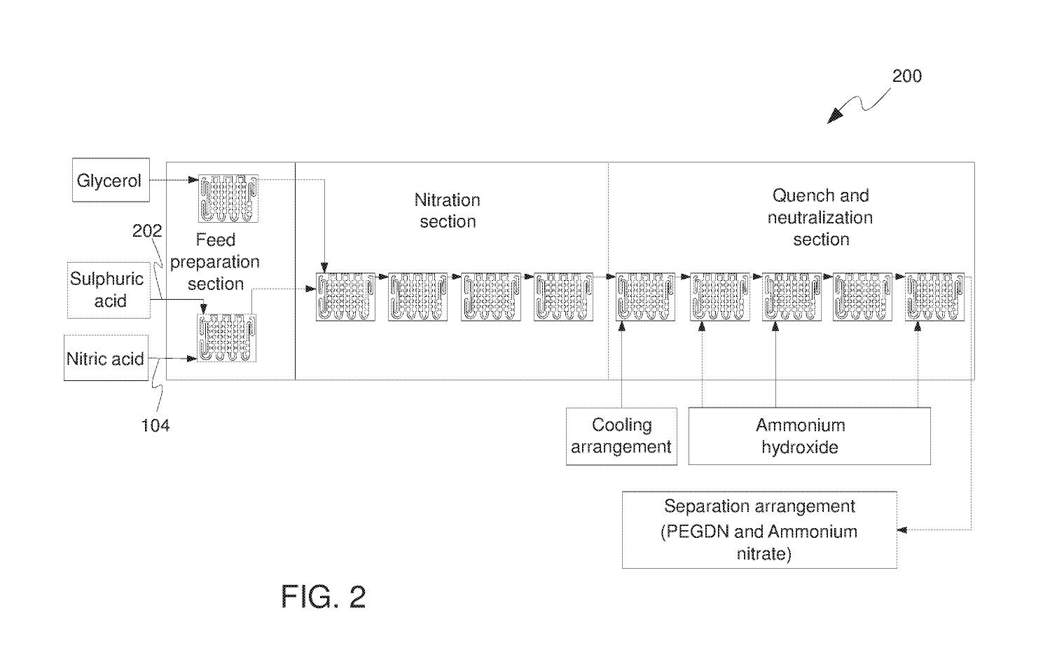

FIG. 2 is an illustration of an apparatus for producing PEGDN and ammonium nitrate, according to another embodiment of the present disclosure; and

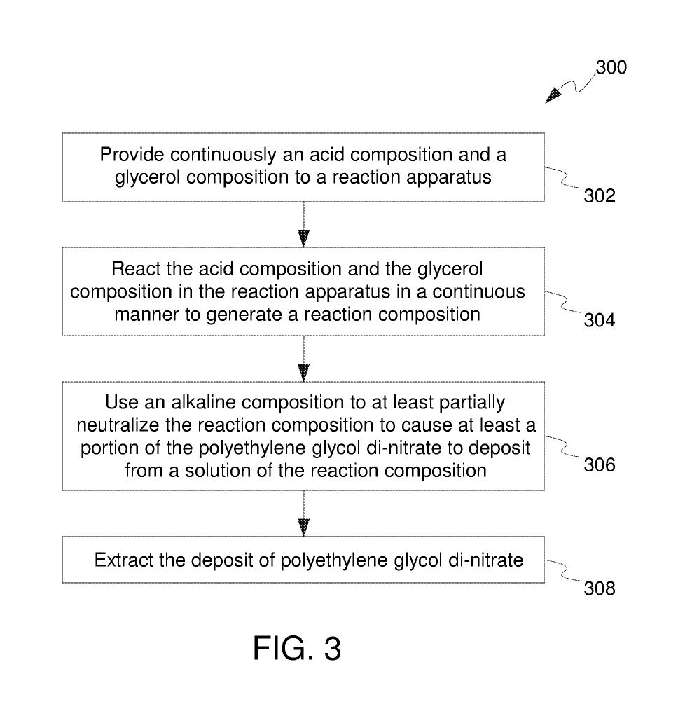

FIG. 3 is an illustration of steps of a method for producing PEGDN and ammonium nitrate, according to an embodiment of the present disclosure.

In the accompanying diagrams, an underlined number is employed to represent an item over which the underlined number is positioned or an item to which the underlined number is adjacent. A non-underlined number relates to an item identified by a line linking the non-underlined number to the item. When a number is nonunderlined and accompanied by an associated arrow, the non-underlined number is used to identify a general item at which the arrow is pointing.

### DESCRIPTION OF EMBODIMENTS OF THE DISCLOSURE

The present disclosure generally relates to an apparatus and a method for production of polyethylene glycol dinitrate (PEGDN), in a continuous manner. Specifically, the apparatus of present disclosure provides improved manufacturing of PEGDN. Furthermore, PEGDN has a chemical formula

O₂N—(O—CH₂—CH₂)ₙONO₂

wherein “n” is an integer indicating the number of (O—CH₂—CH₂) monomer units in the PolyEthylene Glycol DiNitrate (PEGDN) polymer chain.

Generally, production of PEGDN requires an excess of highly concentrated Nitric acid (HNO₃) throughout the reaction; by “highly concentrated” is meant, for example, HNO₃ having a concentration in a range of 75 to 98%. Furthermore, with the progression of the reaction, the concentration of HNO₃ is reduced due to generation of water vapours. Notably, HNO₃ concentration below 85% makes the reaction mixture highly unstable, thereby resulting in a, potentially, violent “fume-off” reaction, simultaneously releasing fumes of Nitrogen Oxides (NOₓ). Therefore, an excess of highly concentrated HNO₃, such as for example 98% HNO₃ is required for ensuring safe continuous production of PEGDN; such an operating condition is achieved in embodiments of the present disclosure.

The PEGDN may be used in conjunction with combustible hydrocarbon fuels. Generally, such apparatus and method employ a process which is based on the below mentioned reactions:

[Aliphatic polyol]+[HNO₃]→Nitrate esters Eq. 1

[Aliphatic polyol]+[HNO₃]+[H₂SO₄]→Nitrate esters Eq. 2

However, the present disclosure is primarily concerned with an improved apparatus and method for (of) producing PEGDN in a continuous manner. The improved method employs a process which is based on the below mentioned reactions:

PEG+[HNO₃]→PEGDN+ammonium nitrate Eq. 3

PEG+[HNO₃]+[H₂SO₄]→PEGDN+ammonium nitrate Eq. 4

Referring now to FIG. 1, shown is an apparatus 100 for producing PEGDN, in a continuous manner, according to an embodiment of the present disclosure. The apparatus 100 comprises a series of reaction cells, such as 106a, 106b,106c, 106d, 106e and 106f; and a separation arrangement 112. The reaction cells, such as 106a, 106b,106c, 106d, 106e and 106f are spatially disposed in one or more planar structures. The plurality of reaction cells, 106a, 106b,106c, 106d, 106e and 106f, include a feed preparation section, a nitration section, and a quench and neutralization section. The apparatus 100 uses dilute nitric acid and PEG for the production of PEGDN. Specifically, the apparatus 100 is configured to mix and react the chosen reagents, to subsequently generate an aqueous stream treated with an alkali, and, finally separate reaction products (PEGDN or another nitrate ester) from the aqueous stream.

As shown, the apparatus 100 includes a feedstream 102 for introducing a glycol composition, such as Polyethylene glycol (PEG), to reaction cells thereof. Optionally, the glycol composition includes PEG with a molecular weight in a range of 150-800. Optionally, general formula for molecular weight or molar mass of PEG is 18.02+44.05n amu or g/mol, wherein “n” is the integer indicating the number of (O—CH₂—CH₂) monomer units of PEG in the PEGDN polymer chain. Specifically, the glycol composition includes PEG molecules with approximate molecular weight in a range of 150 g/mol to 800 g/mol. Therefore, the “n” for the PEG molecules is in a range of 3 to 17. The apparatus 100 also includes another feedstream 104 for introducing an acid composition, such as, for example, a mixture of dilute nitric acid and concentrated sulphuric acid, to reaction cells thereof. Beneficially, handling concentrated nitric acid may be a challenge, in terms of fume-off reactions, high explosive nature, and the like, therefore the present disclosure employs a mixture of dilute nitric acid and concentrated sulphuric acid. The feedstreams 102, 104 along with the reaction cells 106a, 106b constitute a feed preparation section of the apparatus 100. Furthermore, as shown, the reaction cells 106c, 106d constitute a nitration section, and the reaction cells 106e, 106f constitute a quench and neutralization section of the apparatus 100. The acid composition and the glycol composition react in reaction cells, such as 106c, 106d which constitute the nitration section, in a continuous manner to generate a reaction composition. The stoichiometric representation of Equation 2 is provided by Equation 5 (Eq.5) as follows:

H(OCH₂CH₂)ₙOH+2HNO₃+2H₂SO₄→O₂N(OCH₂CH₂)ₙONO₂+H₂O (Eq.5)

It may also be noted that the feed of reaction composition containing polyethylene glycol dinitrate (PEGDN) will contain some water as a result of the esterification reaction between PEG and feed of acid composition containing a mixture of dilute nitric acid and concentrated sulphuric acid. Optionally, the acid composition includes the dilute nitric acid and the concentrated sulphuric acid in equal volumes. Optionally, the acid composition includes the dilute nitric acid in a concentration range of 50 to 70 weight %. For example, the concentration of dilute nitric acid may be in a range from 50, 55, 60 or 65 weight % (as a lower limit) up to 55, 60, 65 or 70 weight % (as an upper limit). Optionally the concentration of dilute nitric acid is 60 weight %. Optionally, the acid composition includes the concentrated sulphuric acid in a concentration range of 96 to 98 weight %. For example, the concentration of concentrated sulphuric acid is optionally in a range from 96, 96.5, 97 or 97.5 weight % (as a lower limit) up to 96.5, 97, 97.5 or 98 weight % (as an upper limit). Optionally, the reacted composition from the nitration section includes, but is not limited to, PEGDN, HNO₃, water and/or impurities. Generally, in the process of recovering PEGDN from the reacted reaction composition, there is employed neutralizing HNO₃ with alkaline composition, wherein heat is released during this process, which increase the risk of fume-off reactions in the reacted mixture.

The quench and neutralization section of the apparatus 100 includes a feed 108 for a cooling arrangement (not shown) for cooling reaction cells to avoid spatial reaction hotspots and thereby preventing thermal runaway occurring within the reaction apparatus, and a plurality of feeds 110a, 110b and 110c for providing an alkaline composition, such as Ammonium hydroxide or ammonia, to at least partially neutralise the reaction composition (i.e. PEG together with nitric acid) to cause at least a portion of the polyethylene glycol dinitrate to deposit from a solution of the reaction composition. Optionally, the feed for the cooling arrangement uses a coolant applied to a region which is spatially adjacent to the series of reaction cells. Optionally, the application of coolant to a region which is spatially adjacent to the series of reaction cells results in exchange of heat, subsequently lowering the temperature of the series of reaction cells. Optionally, the series of reaction cells are cooled in operation using a coolant at a temperature in a range of 0° C. to 15° C. For example, the coolant lowers the temperature of the series of reaction cells in a range from 0, 1, 2, 3, 4, 5, 6, 7, 8, 9, 10, 11, 12, 13 or 14° C. (as a lower limit) up to 1, 2, 3, 4, 5, 10 or 15° C. (as an upper limit). It will be appreciated that controlled temperature of the series of reaction cells ensure blast-free continuous production of PEGDN and avoids any thermal runoffs in the apparatus 100. Alternatively, optionally, the alkaline composition is any one of sodium, potassium, calcium and/or lithium salts, such as for example, sodium carbonate, sodium hydroxide, and so forth. Optionally, the alkaline composition may be employed at the initial stage of the reaction. Optionally, the reaction composition has a pH in a range of 4 to 12. For example, the pH of the reaction composition may be in a range from 4, 5, 6, 7, 8, 9, 10 or 11 (as a lower limit) up to 5, 6, 7, 8, 9, 10, 11 or 12 (as an upper limit).

Following on, the neutralized reaction composition is expelled from the quench and neutralization section of the apparatus 100 to be received by the separation arrangement 112. The separation arrangement 112 separates a biphasic product containing PEGDN and Ammonium Nitrate, in a continuous manner. The separation arrangement 112 is any one of: a thin film evaporator and/or a falling film reactor. Optionally, the separation arrangement 112 execute thermal separation of neutralized reaction composition. In an embodiment, the separation arrangement 112 optionally comprises multiple evaporators connected in series, wherein the neutralized reaction composition from one evaporator may be a feed for the second evaporator in the series of multiple evaporators. Specifically, a thin film, such as of 0.5 mm thick, of neutralized reaction composition is created at a temperature-controlled wall of one of the multiple evaporators, such as a first evaporator. Furthermore, the thin film evaporator separates a volatile component from less volatile components using indirect heat transfer under controlled conditions. Optionally, a continuous feed of steam is provided to the separation arrangement 112 to supply heat required for the evaporation of the feed of the Ammonium Nitrate and water in the reaction composition, thereby separating PEGDN and Ammonium Nitrate. Notably, the volatility of Ammonium Nitrate is higher than that of PEGDN. It will be appreciated that the controlled conditions are potentially most favourable product temperature that increases volatile component stripping and recovery from a biphasic product, such as reduced pressure conditions, vacuum conditions, and the like. For example, the separation arrangement 112 is operated at reduced pressure conditions, to lower the amount of heating required to separate PEGDN and Ammonium Nitrate, thereby lowering the overall temperature thereof.

Optionally, the separation arrangement 112 separates the PEGDN and Ammonium Nitrate using a hydrophobic solvent. The hydrophobic solvent enables substantial evaporation of only Ammonium Nitrate from the biphasic product of the reaction composition. Optionally, the hydrophobic solvent is one of methylene chloride, a hexane, a pentane or a silicone. Notably, methylene chloride, a hexane, a pentane or a silicone evaporate easily in air but do not dissolve in water, thereby, function as non-polar solvents.

Alternatively, the separation arrangement 112 is optioanlly a liquid-liquid separation membrane for isolating PEGDN from the biphasic product of the reaction composition. The liquid-liquid separation membrane is optionally fabricated from functionalized polytetrafluorethylene (PTFE or Teflon™) for the separation of two liquids, PEGDN and Ammonium Nitrate. PTFE is a polymer of carbon and fluorine, and is hydrophobic owing to high electronegativity of fluorine. Furthermore, PTFE is hydrophobic, non-sticky, microporous (frictionless), an excellent insulator, and has a high temperature rating and a high melting temperature. Due to its hydrophobic nature and high temperature rating, PTFE has been used as a coating in utensils, medical devices, graft material in surgeries, and industrial lining in hose assemblies, industrial pipelines, and so forth. Moreover, PTFE is chemically inert and resistant to reactive and corrosive chemicals, such as nitric acid, and is employed in application using handling or storing acids, alkalis or other corrosive chemicals. Therefore, the separation arrangement 112 may separate PEGDN from nitric acid and water, and also remove water and reconcentrate nitric acid.

Furthermore, the frictionless quality of PTFE provides improved flow of highly viscous liquids, even water, therethrough. In an embodiment, the PTFE-based liquid-liquid separation membrane retains one phase of the bi-phasic product, such as PEGDN, on its surface by filling the micropores of the PTFE (referred to as the ‘wetting’ phase) and allows the other phase of the biphasic product, such as water, to pass through it (referred to as the ‘non-wetting’ phase). A pressure differential is applied between the two sides of the PTFE-based liquid-liquid separation membrane to push the wetting phase without forcing the non-wetting phase through the pores of the PTFE-based liquid-liquid separation membrane. Optionally, the pressure differential is applied and adjusted by a pressure controller arranged with the reaction apparatus 100. Notably, the PTFE-based liquid-liquid separation membrane separates two liquids based on the permeate flux and the nitric acid selectivity. Subsequently, the water is allowed to pervaporate to yield concentrated nitric acid. It will be appreciated that the resulting concentrated nitric acid can be recycled back into the feedstream 104 for introducing an acid composition to reaction cells, 106a, 106b,106c, 106d, 106e and 106f. Moreover, the flow rate of lighter hydrophillic component may be in a range of 3 litres per minute (L/min) to 40 litres per minute (L/min). Furthermore, an output 25 tpa to 300 tpa of PEGDN production may be achieved from such PTFE-based liquid-liquid separation membrane. Such a separation arrangement 112 is optionally cost-effective as it potentially replaces the several unit processes of separation of PEGDN and Ammonium Nitrate, and reduce reactant volume, by-products and effluent gas streams. In an embodiment, the reaction apparatus employing a PTFE-based liquid-liquid separation membrane may be operated as a plurality of lines of production (similar to reaction cells, 106a, 106b, 106c, 106d, 106e and 106f), wherein one line in production allows another line to be taken off production for its PTFE-based liquid-liquid separation membranes to be replaced periodically, for example using modular assemblies.

Alternatively, the separation arrangement 112 is optionally a membrane separator that removes impurities and recovers HNO₃. In this example embodiment, the separation process includes using a series of turbo pumps with stainless steel blades. Beneficially, using turbo pumps with stainless steel blades, does not generate heat during recovery of PEGDN, therefore reducing a risk of any explosion occurring.

Alternatively, the separation arrangement 112 optionally comprises an agitator to uniformly separate out the organic (namely, PEGDN) and aqueous (namely, Ammonium Nitrate) phase of the biphasic reaction composition. Furthermore, the separation arrangement 112 may comprise baffle plates to maintain steady state therein. Additionally, organic phase of the biphasic reaction composition, comprising PEGDN, is potentially a heavier phase and potentially settles in the lower portion of the separation arrangement 112. Furthermore, the aqueous phase, comprising Ammonium Nitrate, is potentially separated out continuously from the biphasic reaction composition.

Referring now to FIG. 2, there is shown an apparatus 200 for producing PEGDN, according to another embodiment of the present disclosure. The apparatus 200 of FIG. 2 is substantially structurally and functionally similar to (namely, same as) the apparatus 100 of FIG. 1; however, the apparatus 200 includes a provision for another feedstream 202, for introducing an acid composition comprising concentrated sulphuric acid. Specifically, a feed preparation section of the apparatus 200 includes the feedstream 202 for introducing concentrated sulphuric acid over and above the feedstream 104 (of the apparatus 100) for introducing an acid composition, such as, for example, a mixture of dilute nitric acid and concentrated sulphuric acid, to reaction cells thereof. The feedstream 102 (of the apparatus 100) for introducing a glycol composition and the feedstreams 104 and 202 along with the reaction cells 106a, 106b constitute a feed preparation section of the apparatus 200. Furthermore, the apparatus 200 comprises a nitration section (of the apparatus 100) and a quench and neutralization section (of the apparatus 100). The acid composition, from feedstreams 104 and 202 and the glycol composition react in reaction cells, such as 106c, 106d which constitute the nitration section, in a continuous manner to generate a reaction composition. Following on, the reaction composition is at least partially neutralized using an alkaline composition, such as Ammonium hydroxide or ammonia, to cause at least a portion of the PEGDN to deposit from a solution of the reaction composition. Furthermore, the separation arrangement 112 (of the apparatus 100) separates a biphasic product containing PEGDN and Ammonium Nitrate, in a continuous manner.

The apparatus of FIGS. 1 and 2 are susceptible to being used for manufacturing other types of fuel additives, if required, for example other types of nitrate esters.

Referring now to FIG. 3, there is shown an illustration of steps of a method 300 for producing polyethylene glycol dinitrate (PEGDN) in a continuous manner, in accordance with an embodiment of the present disclosure. Specifically, the method 300 relates to the apparatuses of FIGS. 1 and 2 for the production of PEGDN.

At a step 302, an acid composition and a glycol composition is continuously provided to a reaction apparatus.

At a step 304, the acid composition and the glycol composition react in the reaction apparatus in a continuous manner to generate a reaction composition.

At a step 306, an alkaline composition is used to at least partially neutralize the reaction composition and to cause at least a portion of the polyethylene glycol dinitrate to deposit from a solution of the reaction composition.

At a step 308, the deposit of polyethylene glycol dinitrate is extracted.

The steps 302 to 308 are only illustrative and other alternatives can also be provided where one or more steps are added, one or more steps are removed, or one or more steps are provided in a different sequence without departing from the scope of the claims herein. For example, the method 300 also includes separating the PEGDN and Ammonium Nitrate using a hydrophobic solvent. The method 300 is further explained in detail in conjunction with few examples.

### Example 1 Synthesis and Purification of PEGDN

### Example 1.1 Synthesis of PEGDN Using Continuous Flow and Concentrated Nitric Acid

Example 1.1 mainly corresponds to FIG. 1, in which the preparation of the nitrate ester PEGDN is performed in an advanced flow reactor (such as the apparatus 100). The 100 ml of strong nitric acid (namely, in a range of 80 to 99 weight % or in a range of 96 to 98 weight %) and 100 ml of glycol are mixed together to provide a reaction mixture. The reaction mixture is cooled by using a refrigerated heat transfer fluid, like ethylene glycol for example, applied spatially adjacent to the series of reaction cells to maintain the specified reaction temperature (for example, in a range of 0 to 15° C.). The series of reaction cells, spatially disposed in one or more planar structures in which the acid composition and the glycol composition mix in a turbulent manner determine the purity of the product. The reaction cells are cooled in operation to avoid spatial reaction hotspots and thereby prevent thermal runaway occurring within the reaction apparatus. The neutralization of the acidic reactant composition includes introduction of sufficient Ammonium Hydroxide to affect (namely, to result in) neutralisation of the reaction mixture. The neutralised mixture has a pH in the range of 4 to 12. The two-phase liquid product is then fed into a continuous separation arrangement for receiving the reaction composition and separating therefrom in a continuous manner polyethylene glycol dinitrate (PEGDN) and ammonium nitrate solution. In the separating arrangement the hydrophobic solvent used for extracting the PEGDN is one of methylene chloride and similar polyhalogenated hydrocarbons, a pentane or a hexane. The reagents are, for example, obainable from Fisher Scientific, and are obtainable in at least a 99% degree of purity.

### Example 1.2 Synthesis of PEGDN Using Continuous Flow and Nitric Acid and Concentrated Sulphuric Acid

Example 1.2 corresponds to FIG. 2, i.e. the preparation of the PEGDN uses 50 ml of strong sulphuric acid (i.e., a concentration in a range of 80 to 99 weight % or in a range of 96 to 98 weight %) and dilute nitric acid (i.e. having a concentration in a range of 50 to 70 weight % or 60 weight %) instead of only using the strong nitric acid. Moreover, the preparation of the PEGDN includes use of 50 ml of pure PEG. It will be evident to those skilled in the art that the preparation Example 1.2 follows the same subsequent steps as explained in Example 1.1.

### Example 2 Retrieval of Ammonium Nitrate

Example 2 corresponds to both FIGS. 1 and 2, i.e. the PEGDN separates out of solution during the addition of the ammonium hydroxide as the pH is approaching neutrality. The two-phase liquid product is then fed into a continuous separation arrangement and the PEGDN thus is recovered using a hydrophobic solvent which is one of methylene chloride and similar polyhalogenated hydrocarbons, a hexane, a pentane or a silicone. After such phase separation, two products are obtained. The first product is the required product, namely PEGDN. The second product is a solution of ammonium nitrate, which is a well-known fertiliser.

Furthermore, the aforementioned PEGDN and similar additives can be added to fuels, for example alcohols, heavy fuel oil, LNG, PNG and similar. Such alcohols include, for example: ethanol, methanol.

Alternatively, optionally, other methods for separating PEGDN and Ammonium Nitrate, by using membrane separation technology may be used. The membrane separation technology optionally employ any one of a semi-permeable membrane, a thin film membrane and the like. A driving force for membrane separation is optionally a pressure differential arising in operation between both sides of the membrane. Optionally, the pressure differential is generated at reduced pressure conditions, such as sub-ambient atmospheric pressures. Furthermore, such reduced pressure conditions will result in lowering the amount of heating required to vaporize the Ammonium Nitrate and water, thereby lowering the overall temperature thereof. In view of the above, the reaction apparatus, comprising the series of reaction cells, the feed preparation section, the nitration section, the quench and neutralization section, and the separation arrangement, provides highly purified PEGDN in high yield.

The system and method for producing polyethylene glycol dinitrate (PEGDN), in a continuous manner, of the present disclosure provides many benefits over conventional production methods. The present disclosure employs a continuous process for the production of PEGDN and uses a Ammonium Nitrate in a liquid state. Beneficially, residence time of reactants in the reactor is substantially reduced. Furthermore, use of dillute nitric acid highly increases safety of the process and reduces a risk of thermal runaway and possibility of fume-off reactions. Additionally, the continuous feed of Ammonium Nitrate ensures a uniform heat transfer among the reactants in the reactor. In addition, the process employed in the present disclosure potentially provides an inorganic Ammonium Nitrate salt, as a by-product to the PEGDN, which is highly commercially viable.

Modifications to embodiments of the disclosure described in the foregoing are possible without departing from the scope of the disclosure ______ as defined by the accompanying claims. Expressions such as “including”, “comprising”, “incorporating”, “consisting of”, “have”, “is” used to describe and claim the present disclosure are intended to be construed in a non-exclusive manner, namely allowing for items, components or elements not explicitly described also to be present. Reference to the singular is also to be construed to relate to the plural. Numerals included within parentheses in the accompanying claims are intended to assist understanding of the claims and should not be construed in any way to limit subject matter claimed by these claims.

1. A reaction apparatus for producing polyethylene glycol dinitrate (PEGDN) in a continuous manner, wherein the reaction apparatus comprises:
a series of reaction cells spatially disposed in one or more planar structures, wherein the plurality of reaction cells include:
a feed preparation section having feedstreams for continuously providing an acid composition and a glycol composition to reaction cells thereof, wherein the acid composition includes a mixture of dilute nitric acid and concentrated sulphuric acid,
a nitration section in which the acid composition and the glycol composition react in reaction cells in a continuous manner to generate a reaction composition, and
a quench and neutralization section having a feed for a cooling arrangement for cooling reaction cells to avoid spatial reaction hotspots and thereby preventing thermal runaway occurring within the reaction apparatus, and a plurality of feeds for providing an alkaline composition to at least partially neutralize the reaction composition to cause at least a portion of the polyethylene glycol dinitrate to deposit from a solution of the reaction composition; and
a separation arrangement for separating polyethylene glycol dinitrate (PEGDN) and Ammonium Nitrate, wherein the separation arrangement is a thin film evaporator and/or falling film evaporator.

2. The reaction apparatus of claim 1, wherein the acid composition includes the dilute nitric acid in a concentration range of 50 to 70 weight %.

3. The reaction apparatus of claim 2, wherein the concentration of dilute nitric acid is 60 weight %.

4. The reaction apparatus of claim 1, wherein the acid composition includes the concentrated sulphuric acid in a concentration range of 96 to 98 weight %.

5. The reaction apparatus of claim 1, wherein the glycol composition includes PEG with a molecular weight in a range of 150 to 800.

6. The reaction apparatus of claim 1, wherein the reaction composition has a pH in a range of 4 to 12.

7. The reaction apparatus of claim 1, wherein the separation arrangement separates the PEGDN and Ammonium Nitrate using a hydrophobic solvent.

8. The reaction apparatus of claim 7, wherein the hydrophobic solvent is one of methylene chloride, a hexane, a pentane or a silicone.

9. The reaction apparatus of claim 1, wherein the feed for the cooling arrangement uses a coolant applied to a region which is spatially adjacent to the series of reaction cells.

10. The reaction apparatus of claim 7, wherein the series of reaction cells are cooled in operation using a coolant at a temperature in a range of 0° C. to 15° C.

11. The reaction apparatus of claim 1, wherein the acid composition includes the dilute nitric acid and the concentrated sulphuric acid in equal volumes.
